(19) United States
(12) Patent Application Publication
(10) Pub. No.: US 2006/0120972 A1
(43) Pub. Date: Jun. 8, 2006

(54) 9-(N-methyl-piperidyliden-4)-thioxanthene for treatment of pulmonary hypertension

(76) Inventors: Peter Engels, Gladbach (DE); Beate Schmitz, Koln (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/255,567

(22) Filed: Oct. 21, 2005

Related U.S. Application Data
(60) Provisional application No. 60/626,249, filed on Nov. 9, 2004.

(51) Int. Cl. A61K 31/453; A61K 9/14; A61K 9/70; A61L 9/04

(52) U.S. Cl. 424/46; 514/320; 424/449

(57) ABSTRACT

Pimethixene is used as a medicament for the treatment of pulmonary hypertension.

### CROSS-REFERENCE TO RELATED APPLICATION

This application claims priority under 35 U.S.C. § 119(e) to U.S. Provisional Application No. 60/626,249, filed Nov. 9, 2004, which is entirely incorporated herein by reference.

### BACKGROUND OF THE INVENTION

1. Field of the Invention

The present invention relates to the use of 9-(N-methyl-piperidyliden-4)-thioxanthene as a medicament for the treatment of diseases aggravated by increased levels of 5-HT and mediated by 5HT_{2B} receptors.

2. Description of the Related Art

Serotonin, a neurotransmitter with mixed and complex pharmacological characteristics, was first discovered in 1948, and subsequently has been the subject of substantial research. Serotonin, also referred to as 5-hydroxytryptamine (5-HT), acts both centrally and peripherally on discrete 5-HT receptors. Currently, fourteen subtypes of serotonin receptor are recognized and delineated into seven families, 5-HT₁ to 5-HT₇. Within the 5-HT₂ family, 5-HT_{2A}, 5-HT_{2B} and 5-HT_{2C} subtypes are known to exist. These subtypes share sequence homology and display similarities in their specificity for a wide range of ligands. Nomenclature and classification of 5-HT receptors have been reviewed (see Martin and Humphrey, Neuropharm. 1994, 33, 261-273 and Hoyer et al., Pharm. Rev. 1994, 46, 157-203).

The 5-HT_{2B} receptor, initially termed 5-HT_{2F}, or serotonin receptor like (SRL), was first characterized in rat isolated stomach fundus (see Clineschmidt et al., J. Pharmacol. Exp. Ther. 1985, 235, 696-708; Cohen and Wittenauer, J. Cardiovasc. Pharmacol. 1987, 10, 176-181) and initially cloned from rat (see Foguet et al., EMBO 1992, 11, 3481-3487) followed by the cloning of the human 5-HT_{2B} receptor (see Schmuck et al., FEBS Lett. 1994, 342, 85-90; Kursar et al., Mol. Pharmacol. 1994, 46, 227-234). The closely related 5-HT_{2C} receptor, widely distributed in the human brain, was first characterized as a 5-HT_{1C} subtype (see Pazos et al., Eur. J. Pharmacol. 1984, 106, 539-546) and was subsequently recognized as belonging to the 5-HT₂ receptor family (see Pritchett et al., EMBO J. 1988, 7, 4135-4140).

Because of the similarities in the pharmacology of ligand interactions at 5-HT_{2B} and 5-HT_{2C} receptors, many of the therapeutic targets that have been proposed for 5-HT_{2C} receptor antagonists are also targets for 5-HT_{2B} receptor antagonists. Current evidence strongly supports a therapeutic role for 5-HT_{2B/2C} receptor antagonists in treating anxiety (e.g., generalized anxiety disorder, panic disorder and obsessive compulsive disorder), alcoholism and addiction to other drugs of abuse, depression, migraine, steep disorders, feeding disorders (e.g., anorexia nervosa) and priapism. Additionally, current evidence strongly supports a therapeutic role for selective 5-HT_{2B} receptor antagonists that will offer distinct therapeutic advantages collectively in efficacy, rapidity of onset and absence of side effects. Such agents are expected to be useful in the treatment of hypertension, disorders of the gastrointestinal track (e.g., irritable bowel syndrome, hypertonic lower esophageal sphincter, and motility disorders), restenosis, asthma and obstructive airway disease, and prostatic hyperplasia (e.g., benign prostatic hyperplasia).

EP 1 270 568 and WO 03/35646 describe selective 5HT_{2B} receptor antagonists which are derivatives of 4-(thio- or selenoxanthene-9-ylidene)-piperidine or acridine having in any case several side groups in the aromatic ring system.

The involvement of 5-HT receptor activation in the development of pulmonary hypertension has been described by M. MacLean in TiPS, vol. 20, pg. 490-495 (1999). However, in this review article, the disorder state is referred to activation of 5HT_{ID/1B} and 5HT_{2A} receptors.

J.-M. Launay et al. describe in Nature Medicine Vol. 8, No. 10 (2002), p. 1129-1135 that serotonin as well as other agonists of the 5-HT_{2B} receptor support the development of pulmonary hypertension. Further, the study showed that 5HT_{2B} receptor expression is upregulated in pulmonary hypertension.

Pimethixene is the common name of 9-(N-methyl-piperidyliden-4)-thioxanthene. Pimethixene was developed in the 1960's and has been used for the treatment of sleep disorders, hyperactivity, anxiety, allergy, as bronchodilator, and for anesthesia.

### SUMMARY OF THE INVENTION

Preferred embodiments provide for a method of treating a disease aggravated by increased levels of 5-HT comprising administering to a subject a compound according to the formula: 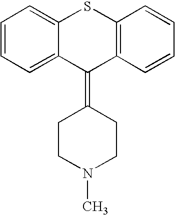
or a pharmaceutically acceptable salt thereof.

### DETAILED DESCRIPTION OF THE PREFERRED EMBODIMENT

The preferred embodiments provide a new medicament comprising a compound effective for treatment of disease aggravated by increased levels of 5-HT in blood or blood vessels, such as, but not limited to, pulmonary hypertension.

The preferred embodiments provide the use of a compound according to formula: 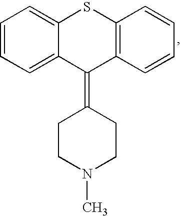
also called pimethixene, or a pharmaceutically acceptable salt thereof for the development of a medicament for the treatment of disease aggravated by increased levels of 5-HT, such as, but not limited to, pulmonary hypertension.

The preferred embodiments further relate to a pharmaceutical composition comprising such a compound or a pharmaceutically acceptable salt thereof, in admixture with one or more pharmaceutically acceptable carriers.

“Pharmaceutically acceptable salt” refers to those salts which retain the biological effectiveness and properties of the free bases and which are not biologically or otherwise undesirable, formed with inorganic acids such as, but not limited to, hydrochloric acid, hydrobromic acid, sulfuric acid, nitric acid, phosphoric acid and the like, and organic acids such as, but not limited to, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, ascorbic acid and the like.

The term “treatment” as used herein covers any treatment of a disease in a mammal, particularly a human, and includes:

(i) preventing the disease from occurring in a subject which may be predisposed to the disease but has not yet been diagnosed as having it;

(ii) inhibiting the disease, i.e., arresting its development; or

(iii) relieving the disease, i.e., causing regression of the disease.

The term “disease state”, “disease”, or “disease aggravated by increased levels of 5-HT” as used herein is intended to cover all disease states which are mediated by 5-HT₂ receptors due to increased serotonin level and/or plasma protein extravasation from the blood into the surrounding tissue. Such disease states include, but are not limited to, pulmonary hypertension.

Pulmonary hypertension is an abnormal elevation of the pressure in the blood vessels of the lungs, also called “high blood pressure” of the lungs. In normal lungs, the pressure in the blood vessels is about one-quarter of the pressure in the arteries of the body and can temporarily adapt to increased pressures that occur during exercise. In pulmonary hypertension, the small arteries in the lungs are too narrow, so the pressure rises in these vessels. As a result, the right side of the heart, which pumps blood into the lungs, has to pump against a higher resistance to blood flow. This makes it more difficult to pump the blood through the lungs, particularly when increased flow is needed, as when a subject exercises.

The compound of the preferred embodiments has been found to be a human 5-HT_{2B} receptor antagonist. Affinity for the 5-HT_{2B} receptors was demonstrated using an in vitro binding assay utilizing cloned human 5-HT_{2B} receptors radiolabelled with [³H]-5HT, as well as luciferase reporter gene assays as shown in the examples. Antagonist properties for the human 5-HT_{2B} receptor were shown by counter screening at human 5-HT_{2A} and 5-HT_{2C} receptors. Affinity for the human H₁ receptor was determined using an in vitro binding assay utilizing cloned human H₁ receptors radiolabelled with [³H]-mepyramine and antagonistic properties were determined by a luciferase reporter gene assay, as shown in the examples.

Accordingly, the compound of the preferred embodiments is useful for treating diseases which can be ameliorated by blockade of 5-HT₂ and in particular 5HT_{2B} receptors. Because of the similarities in the pharmacology of ligand interactions at 5-HT_{2C} and 5-HT_{2B} receptors, many of the therapeutic targets that have been proposed for 5-HT_{2C} receptor antagonists are also targets for 5-HT_{2B} receptor antagonists.

Experimental evidence supports a therapeutic role for 5-HT_{2B} receptor antagonists in treating pulmonary hypertension. In pulmonary hypertension, one of the most profound increases in vascular responsiveness is observed for serotonin. Two lines of evidence imply that this results from a switch in the receptor mediating vasoconstriction from predominantly 5-HT_{2A} to predominantly 5-HT_{2B}. First, serotonin induced contractions of isolated blood vessels from hypertensive animals become resistant to block by selective 5-HT_{2A} receptor antagonists, but remain sensitive to non-selective 5-HT_{2B} receptor antagonists. Second, there is an increase in 5-HT_{2B} receptor mRNA in vessels from hypertensive animals (see Watts et al., J. Pharmacol. Exp. Ther. 1996, 277, 1103-13 and Watts et al., Hypertension 1995, 26, 1056-1059). This hypertension-induced shift in the population of receptor subtype mediating constrictor responses to 5-HT suggests that selective block of vasoconstrictor 5-HT_{2B} receptors may be of therapeutic benefit in the treatment of pulmonary hypertension.

Pharmaceutical Compositions

In applying the compound of the preferred embodiments to treatment of the above conditions, administration of the active compound and salts described herein can be via any of the accepted modes of administration, including inhalation, oral, parenteral and otherwise systemic route of administration. Any pharmaceutically acceptable mode of administration can be used, including solid, semi-solid or liquid dosage forms, such as, for example, tablets, suppositories, pills, capsules, powders, liquids, suspensions, aerosols or the like, preferably in unit dosage forms suitable for single administration of precise dosages, or in sustained or controlled release dosage forms for the prolonged administration of the compound at a predetermined rate. The compositions will typically include a conventional pharmaceutical carrier or excipient and at least the compound of the preferred embodiments or one of the pharmaceutically acceptable salts thereof and, in addition, may include other medicinal agents, pharmaceutical agents, carriers, adjuvants, etc.

The amount of the compound of the preferred embodiments administered can be dependent on the subject being treated, the severity of the affliction, the manner of administration and the judgment of the prescribing physician. However, an effective dose for inhalation, oral, parenteral and otherwise systemic routes of administration is in the range of about 0.01 to about 20 mg/kg/day, preferably about 0.1 to about 10 mg/kg/day. For an average 70 kg human, the dose would be about 0.7 to about 1400 mg per day, or preferably about 7 to about 700 mg/day.

One of ordinary skill in the art of treating such diseases will be able, without undue experimentation and in reliance upon personal knowledge and the disclosure herein, to ascertain a therapeutically effective amount of the compound for a given disease.

For solid compositions, conventional non-toxic solid carriers include, for example, pharmaceutical grades of mannitol, lactose, cellulose, cellulose derivatives, sodium crosscarmeillose, starch, magnesium stearate, sodium saccharin, talcum, glucose, sucrose, magnesium carbonate, and the like may be used. The active compound as defined above may be formulated as suppositories using, for example, polyalkylene glycols, e.g. PEG (polyethyleneglycol) or PEG derivatives, acetylated triglycerides and the like, as the carrier. Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. The composition or formulation to be administered will, in any event, contain a quantity of the active compound(s) in an amount effective to alleviate the symptoms of the subject being treated.

Pimethixene or a pharmaceutically acceptable salt thereof may be formulated in a suitable form for administration by inhalation (e.g. via an aerosol) or insufflation (either through the mouth or nose).

One manner for administering a compound of the preferred embodiments is inhalation. Inhalation is an effective means for delivering an agent directly to the respiratory tract. There are three general types of pharmaceutical inhalation devices: nebulizer inhalers, dry powder inhalers (DPI), and metered-dose inhalers (MDI). Nebulizer devices produce a stream of high velocity air that causes a therapeutic agent to spray as a mist which is carried into the patient's respiratory tract. The therapeutic agent is formulated in a liquid form such as a solution or a suspension of micronized particles of respirable size, where micronized is typically defined as having about 90% or more of the particles with a diameter of less than about 10 μm. A typical formulation for use in a conventional nebulizer device is an isotonic aqueous solution of a pharmaceutical salt of the active agent at a concentration of the active agent of between about 0.05 μg/mL and about 10 mg/mL.

DPI's typically administer a therapeutic agent in the form of a free flowing powder that can be dispersed in a patient's air-stream during inspiration. In order to achieve a free flowing powder, the therapeutic agent can be formulated with a suitable excipient, such as lactose or starch. A dry powder formulation can be made, for example, by combining dry lactose having a particle size between about 1 μm and about 100 μm with micronized particles of a pharmaceutical salt of the active agent and dry blending. Alternative, the agent can be formulated without excipients. The formulation is loaded into a dry powder dispenser or into inhalation cartridges or capsules for use with a dry powder delivery device.

Examples of DPI delivery devices provided commercially include Diskhaler (GlaxoSmithKline, Research Triangle Park, N.C.) (see, e.g., U.S. Pat. No. 5,035,237); Diskus (GlaxoSmithKliine) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI's typically discharge a measured amount of therapeutic agent using compressed propellant gas. Formulations for MDI administration include a solution or suspension of active ingredient in a liquefied propellant. While chlorofluorocarbons, such as CCl₃F, conventionally have been used as propellants, due to concerns regarding adverse affects of such agents on the ozone layer, formulations using hydrofluoroalkanes (HFA), such as 1,1,1,2-tetrafluoroethane (HFA 134a) and 1,1,1,2,3,3,3,-heptafluoro-n-propane, (HFA 227) have been developed. Additional components of HFA formulations for MDI administration include co-solvents, such as ethanol or pentane, and surfactants, such as sorbitan trioleate, oleic acid, lecithin, and glycerin. (See, for example, U.S. Pat. No. 5,225,183, EP 0717987 A2, and WO 92/22286.)

Thus, a suitable formulation for MDI administration can include from about 0.01% to about 5% by weight of a pharmaceutical salt of active ingredient, from about 0% to about 20% by weight ethanol, and from about 0% to about 5% by weight surfactant, with the remainder being the HFA propellant. In one approach, to prepare the formulation, chilled or pressurized hydrofluoroalkane is added to a vial containing the pharmaceutical salt of active compound, ethanol (if present) and the surfactant (if present). To prepare a suspension, the pharmaceutical salt can be provided as micronized particles. The formulation is loaded into an aerosol canister, which forms a portion of an MDI device. Examples of MDI devices developed specifically for use with HFA propellants are provided in U.S. Pat. Nos. 6,006,745 and 6,143,277.

In an alternative preparation, a suspension formulation is prepared by spray drying a coating of surfactant on micronized particles of a pharmaceutical salt of active compound. (See, for example, WO 99/53901 and WO 00/61108.) For additional examples of processes of preparing respirable particles, and formulations and devices suitable for inhalation dosing see U.S. Pat. Nos. 6,268,533, 5,983,956, 5,874,063, and 6,221,398, and WO 99/55319 and WO 00/30614.

With regard to construction of the delivery device, any form of aerosolization known in the art, including but not limited to nebulization, atomization or pump aerosolization of a liquid formulation, and aerosolization of a dry powder formulation, can be used in the practice of the preferred embodiments. A delivery device that is uniquely designed for administration of solid formulations is envisioned. Often, the aerosolization of a liquid or a dry powder formulation will require a propellent. The propellent can be any propellent generally used in the art. Examples of useful propellents include chlorofluorocarbons, hydrofluorocarbons, hydrochlorofluorocarbons, and hydrocarbons, including trifluoromethane, dichlorodifluoroethane, dichlorotetrafluoroethanol, and 1,1,1,2-tetrafluoroethane, and combinations thereof.

Systems of aerosol delivery, such as the pressurized metered dose inhaler and the dry powder inhaler are disclosed in Newman, Aerosols and the Lung, Clarke, S. W. and Davia, D. editors, pp 197-22 and may be used in connection with the preferred embodiments.

Dosage forms or compositions containing pimethixene compound or a pharmaceutically acceptable salt thereof in the range of about 0.25 to about 95% by weight with the balance made up from non-toxic carrier may be prepared.

For oral administration, a pharmaceutically acceptable non-toxic composition is formed by the incorporation of any of the normally employed excipients, such as, for example pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain about 1 to about 95% by weight of a compound of the preferred embodiments, more preferably about 2 to about 50% by weight, most preferably about 5 to about 8% by weight.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

A more recently devised approach for parenteral administration employs the implantation of a slow-release or sustained-release system, such that a constant level of dosage is maintained (see, e.g., U.S. Pat. No. 3,710,795).

The percentage of active compound contained in such parenteral compositions is highly dependent on the specific nature thereof, as well as the activity of the compound and the needs of the subject. However, percentages of pimethixene or a pharmaceutically acceptable salt thereof of about 0.1 to about 10% by weight in solution are employable, and will be higher if the composition is a solid which will be subsequently diluted to the above percentages. Preferably, the composition comprises about 0.2 to about 2% by weight of pimethixene or a pharmaceutically acceptable salt thereof in solution.

Transdermal or “pulsed” transdermal administration may be supported by cremes, gels, dispersions and the like.

The composition of the preferred embodiments may also be formulated for administration in any convenient way by analogy with other topical compositions adapted for use in mammals. These compositions may be presented for use in any conventional manner with the aid of any of a wide variety of pharmaceutical carriers or vehicles. For such topical administration, a pharmaceutically acceptable non-toxic formulation can take the form of semisolid, liquid, or solid, such as, for example, gels, creams, lotions, solutions, suspensions, ointments, powders, or the like. As an example, the active component may be formulated into a syrup or gel using ethanol, propylene glycol, propylene carbonate, polyethylene glycols, diisopropyl adipate, glycerol, water, etc., with appropriate gelling agents, such as Carbomers, Klucels, etc. If desired, the formulation may also contain minor amounts of non-toxic auxiliary substances such as preservatives, antioxidants, pH buffering agents, surface active agents, and the like. Actual methods of preparing such dosage forms are known, or will be apparent, to those skilled in the art; for example, see Remington's Pharmaceutical Sciences, Mack Publishing Company, Easton, Pa., 19th Edition, 1995.

Preferably the pharmaceutical composition is administered in a single unit dosage form for continuous treatment or in a single unit dosage form ad libitum when relief of symptoms is specifically required.

### EXAMPLES

Example 1

### Cloned Human 5-HT_{2B} Receptor Binding Assay

The following describes an in vitro binding assay utilizing cloned 5-HT_{2B} receptors radiolabelled with [³H]-5HT.

Receptor Binding Assay

HEK 293 cells transiently transfected with an expression plasmid pXMD1-hu2B encoding the human 5-HT_{2B} receptor (see Schmuck et al., FEBS Left., 1994, 342, 85-90) were used as described previously (Schmuck et al., Eur. J. Pharmacol., 1996, 8, 959-967). Two days after transfection cells were harvested, pelleted at 500 g for 5 min at 4° C., gently resuspended in ice-cold buffer1 (50 mM TRIS pH 7.7, 4 mM CaCl₂) and homogenized using a Polytron PT 1200 tissue homogenizer (position 6 for 30 s). Cells were pelleted at 50,000 g, 4° C. for 10 min, washed with buffer and pelleted again. The final pellet was resuspended in incubation buffer (50 mM TRIS pH 7.7, 4 mM CaCl₂, 10 μM pargyline and 0.1% by weight ascorbic acid). The binding assay consisted of 300 μl of membrane suspension (protein concentration=0.3 to 0.5 mg/ml), 150 μl of competing drug and 50 μl of [³H]5-HT at a final concentration of 4 to 5 nM. The mixture was incubated at 37° C. for 30 min and the assay terminated by rapid filtration and two washing steps with 5 ml of cold 20 mM Iris-NCI pH=7.5, and 154 mM NaCl over Whatman GFB filters. Filters were counted by liquid scintillation. Non-specific binding was determined in the presence of an excess of 5-HT (100 μM). Bound radioligand represented less than 1% of free radioligand. In competition experiments, specific binding represented about 60% of total binding. Results expressed as pKᵢ values are shown in Table 1

### Example 2

### 5-HT_{2A} and 5-HT_{2C} Receptor Binding Methods

The following describes receptor binding methods in which the ligand with high affinity for 5-HT_{2B} receptors were counter screened at 5-HT_{2A} and 5-HT_{2C} receptors to demonstrate selectivity.

5-HT_{2A} receptors were labelled with [³H]ketanserin in human cortex, in HEK293 cells expressing a cloned human 5-HT_{2A} receptor and in HEK293 cells expressing the rat 5-HT_{2A} receptor. For competition binding studies the ligand concentration was approximately 0.1 nM. For saturation binding studies concentrations of radioligand ranged from 0.01 nM to 2.0 nM. Assays were conducted in 0.5 ml of assay buffer (50 mM Tris-HCl, 4 mM calcium chloride, 0.1% by weight ascorbic acid) (pH 7.4 at 4° C.). Non-specific binding was defined with 10 mM unlabelled ketanserin. After 60 min incubation at 32° C., membranes were harvested onto filters treated with 0.1% by weight of polyethylenimine and the bound radioactivity was determined.

Human 5-HT_{2B} receptors were labelled in HEK293 cells as described above except that the radioligand was [³H]-5HT and that the assay buffer contained pargyline in a concentration of 10 mM and 0.1% by weight of ascorbic acid. For competition binding studies the radioligand concentration was approximately 0.4 nM while for saturation binding studies the concentration of [³H]-5HT ranged from 0.05 to 8 nM. Non-specific binding was defined with 10 mM 5-HT. Incubations were for 120 min at 4° C.

5-HT_{2C} receptors were labelled in Cos-7 cells expressing the human 5-HT_{2C} receptor and in NIH-3T3 expressing the rat 5-HT_{2C} receptor.

Assays were conducted as described for the 5-HT_{2A} receptor except that the radioligand was [³H]mesulergine. The radioligand concentration for competition studies was approximately 0.2 nM while for saturation binding studies the concentration ranged from 0.1 to 18 nM. Non-specific binding was defined with 10 μM unlabelled mesulergine.

Competition radioligand binding data were analyzed using a four parameter logistic equation and iterative curve-fitting techniques to obtain estimates of the IC₅₀ and Hill slope. Kd values, determined from saturation binding studies were then used to calculate inhibition dissociation constants (Ki).

Proceeding as in the example above the compound of the preferred embodiments was found to have selective affinity for the 5-HT_{2B} receptor versus 5HT_{2C} receptor. Results are shown in Table 1

### Example 3

### Cloned Human H₁, H₂, and H₃ Receptor Binding Assay

CHO cells were stably transfected with an expression plasmid pCineohH1, pCineohH2, pCineohH3 and pCineohH4 encoding the human Histamine H₁, H₂, H₃ or H₄ receptor, respectively. Recombinant cell lines were harvested after confluent growth, homogenized in ice-cold 50 mM Na₂/potassium phosphate buffer (pH 7.4) and used for radioligand binding studies. Cell homogenates (40-50 μg of protein) were incubated for 30 min at 25° C. in 50 mM Na₂/potassium phosphate buffer (pH 7.4) in 400 μl with the various concentrations of either [³H]-mepyramine, [³H]-thiotidine, [³H]-R-a-Methylhistamine, and [³H]-pyramilamine for cells expressing recombinant human H₁, H₂, H₃ and H₄ receptors, respectively. The nonspecific binding was defined in the presence of 1 μM mianserin. In displacement studies, cell homogenated were incubated either with 1 nM [³H]-mepyramine, 15 nM [³H]-thiotidine, 0.5 nM [³H]-R-α-Methylhistamine, or 15 nM [³H]-pyramilamine and increasing concentrations of competing ligands. The incubations were stopped by rapid dilution with 3 ml of ice-cold 50 mM Na₂/potassium phosphate buffer (pH 7.4). The bound radioactivity was separated by filatration through Whatman GF/C filters that had been treated with 0.3% polyethyleneimine. Filters were washed twice in 3 ml of buffer and radioactivity retained on the filters was measured by liquid scintillation counting.

The concentration of Pimethixene producing 50% inhibition of binding (IC₅₀) was determined using iterative curve fitting techniques.

Proceeding as in the example above, the compound of the preferred embodiments was found to have no affinity for the human H_{2,3} and H4 receptor while it showed high affinity to the H1 receptor.

TABLE 1
RECEPTOR AFFINITY: PK VALUES OF THE PIMETHIXENE

| | Pimethixene | Methysergide | Pizotifen |
|---|---|---|---|
| Receptor | pKi values | | |
| 5HT_{2A} | 10.2 | 8.54 | |
| 5HT_{2B} | 10.6 | 8.85 | 8.54 |
| 5HT_{2C} | 8.6 | | |
| H₁ | 11.1 | | |
| H₂ | low activity | | |
| H₃ | low activity | | |
| H₄ | low activity | | |
| M₁ | 8.6 | | |
| D₂ | 8.2 | | |
| Alpha _{1A} | 7.6 | | |

Summary of pKi values of Pimethixene for the respective receptor subtypes. pKi values were calculated using the equation of Cheng and Prusoff {pKi−log (IC₅₀/[l+used concentration of Agonist/EC₅ₒ])}. EC₈₀ values were calculated from corresponding dose-response experiment using 5-HT, histamine, epinephrine (alpha 1A adrenergic receptor), dopamine (D2 dopaminergic receptor), and acetylcholine (M1 muscarinic receptor) as agonists.

The results of Table 1 show that the compound of the preferred embodiments has a selective affinity for the 5HT_{2A/B} and H₁ receptor, particularly have a much higher affinity for the 5HT_{2B} receptor than for the 5HT_{2C} receptor or other monoaminergic receptors.

### Example 4

### Functional Test of Pimethixene in the mCPP Model in Guinea Pigs

In accordance to the protocol published by Markowitz et. al; (1987) and Olesen et. al; (2000), guinea pigs were used to determine the amount of plasma protein released at the blood vessels within the Dura mater after mCPP application.

After deep anesthesia the test compounds pimethixene, methysergide or pizotifen were injected intravenous in the Vena jugularis at different concentrations. 2 minutes later 1 μg/kg body weight mCPP were injected and followed by the i.v. application of EvansBlue as marker for the protein plasma extravasation. 15 minutes later animals were transcranial perfused and decapitated. A defined area of the dura mater was isolated and transferred into an eppendorf tube containing 200 μl formamid. Because EvansBlue binds to plasma protein, the amount of bound marker dye is a measure of extravasation of plasma protein. Formamid dissolves EvansBlue from the protein which can be calorimetric detected and quantified at 600 nm.

TABLE 2
DETERMINATION OF PLASMA PROTEIN EXTRAVASATION
AS EVANSBLUE EXTINCTION AT 600 NM.

| Dose | | | | | | |
|---|---|---|---|---|---|---|
| in | | | | | | |
| μg/kg | Pimethixene | n | Pizotifen | n | Methysergide | n |
| 100 | not determined | — | 0.230 ± 0.018 | 7 | 0.189 ± 0.026 | 3 |
| 10 | not determined | — | 0.144 ± 0.035 | 8 | 0.228 ± 0.064 | 5 |
| 1 | not determined | — | 0.404 ± 0.025 | 6 | 0.250 ± 0.027 | 5 |
| 0.1 | 0.126 ± 0.028 | 5 | not determined | — | 0.265 ± 0.034 | 5 |
| 0.01 | 0.157 ± 0.040 | 4 | not determined | — | not determined | — |
| 0.001 | 0.269 ± 0.022 | 5 | not determined | — | not determined | — |

An extinction of 0,300±0,038 was determined for the positive control (mCPP agonist), an extinction of 0,130±0,024 was measured for the negative control.

As shown in Table 2, pimethixene inhibited the plasma protein extravasation at concentrations 100 to 1000 fold lower than the reference compounds methysergide or pizotifen.

The references cited herein are entirely incorporated herein by reference.

Many modifications and variations of the embodiments described herein may be made without departing from the scope, as is apparent to those skilled in the art. The specific embodiments described herein are offered by way of example only.

What is claimed is:

1. A method of treating pulmonary hypertension comprising administering to a subject a compound according to the formula: 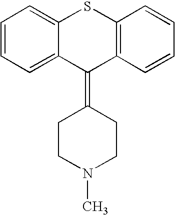
or a pharmaceutically acceptable salt thereof.

2. The method according to claim 1, wherein the administration is performed by a mode selected from the group consisting of inhalation, oral, parenteral, systemic, transdermal, and topical.

3. The method according to claim 2, wherein the administration is by inhalation.

4. The method according to claim 3, wherein the inhalation is performed with a nebulizer inhaler, a dry powder inhaler, or a metered dose inhaler.

5. The method according to claim 2, wherein the administration is by oral administration.

6. The method according to claim 5, wherein the oral administration is performed with a form selected from the group consisting of solution, suspension, tablet, pill, capsule, powder, and sustained release formulations.

7. The method according to claim 6, wherein the form comprises about 1 to about 95% by weight of the compound.

8. The method according to claim 7, wherein the form comprises about 2 to about 50% by weight of the compound.

9. The method according to claim 2, wherein the administration is by parenteral administration.

10. The method according to claim 9, wherein parenteral administration is performed with a solution of about 0.1 to about 10% by weight of the compound.

11. The method according to claim 2, wherein the administration is topical or transdermal.

12. The method according to claim 11, wherein administration is performed with a form selected from the group consisting of gel, cream, lotion, solution, suspension, ointment, powder, and dispersion.

13. The method according to claim 1, wherein the compound is administered at a dose of about 0.01 to about 20 mg/kg/day.

14. The method according to claim 13, wherein the compound is administered at a dose of about 0.1 to about 10 mg/kg/day.
